(19) United States
(12) Patent Application Publication
(10) Pub. No.: US 2025/0217539 A1
(43) Pub. Date: Jul. 3, 2025

(54) SYSTEMS AND METHODS FOR A LARGE LANGUAGE MODEL-BASED SAFE USAGE PLAN GENERATOR FOR HUMAN-IN-THE-LOOP CYBER-PHYSICAL SYSTEMS

(71) Applicants: Sandeep Gupta, Phoenix, TX (US); Ayan Banerjee, Gilbert, AZ (US)

(72) Inventors: Sandeep Gupta, Phoenix, TX (US); Ayan Banerjee, Gilbert, AZ (US)

(73) Assignee: Arizona Board of Regents on Behalf of Arizona State University, Tempe, AZ (US)

(21) Appl. No.: 19/003,399

(22) Filed: Dec. 27, 2024

Related U.S. Application Data
(60) Provisional application No. 63/615,231, filed on Dec. 27, 2023.

(51) Int. Cl. G06F 30/17; G06F 40/30; G06N 5/04; G06Q 50/26; G16H 20/10; G16H 20/60

(52) U.S. Cl. CPC G06F 30/17; G06N 5/04; G06F 40/30; G06Q 50/265; G16H 20/10; G16H 20/60

(57) ABSTRACT

Example implementations of a neural network implementing a large language model for generating action plans that align with physical system dynamics of a cyber-physical system (CPS) but are also safe for the human users are disclosed. Examples include a physical dynamics coefficient estimator based on a liquid time constant neural network that can derive coefficients of dynamical models with some unmeasured state variables. Further, the model coefficients are then used to train an LLM with prompts embodied with traces from dynamical system and the corresponding model coefficients. When integrated with a contextualized chatbot, feasible and safe plans can be generated to manage external events such as meals for automated insulin delivery systems used by Type 1 Diabetes subjects.

### CROSS REFERENCE TO RELATED APPLICATIONS

This is a non-provisional patent application that claims benefit to U.S. Provisional Patent Application Ser. No. 63/615,231 filed on Dec. 27, 2023, which is herein incorporated by reference in its entirety.

### FIELD

The present disclosure generally relates to human-in-the-loop cyber-physical systems, and in particular, to a system and associated methods for applying large language models (LLM) to human-in-the-loop human-in-the-plant cyber physical systems (CPS) to translate high level prompts into action plans that align with the physical system dynamics of the CPS and are also safe for the human users.

### BACKGROUND

Safety criticality implies that operation of an autonomous cyber physical system (CPS) has the potential to harm human participants who are affected by the CPS goal.

Given the impending risks to the human user, safety critical applications often operate with a human in the loop (HIL) system. In such systems, the human is in charge of starting and stopping automation and can provide manual inputs when safety concerns or operational inefficiencies are perceived. In medical applications such as automated insulin delivery, this system results in a human in the loop-human in the plant (HIL-HIP) system model.

The HIP component results in complex dynamical systems such as biological or biochemical processes, with hard requirements on the safety criteria that must be satisfied under all circumstances. Moreover, the HIP components contribute to increased variability and uncertainty in the plant dynamics compared to CPS without HIP.

It is with these observations in mind, among others, that various aspects of the present disclosure were conceived and developed.

### BRIEF DESCRIPTION OF THE DRAWINGS

FIG. 1 is an illustration of a human in the loop human in the plant autonomous cyber physical system (CPS).

FIG. 2 is a diagram illustrating operation of a computer-implemented method for operating the CPS-LLM described herein to, e.g., generate a safe CPS usage plan.

FIG. 3 is a diagram illustrating a liquid time constant network encoder decoder architecture for dynamics coefficient extraction.

FIG. 4 is a set of time series generated by CPS-LLM and the RSME with respect to simulation results.

FIG. 5 is a report of safe glucose results for three approaches of generating a CPS usage plan: (1) manual, (2) using an untuned LLM, and (3) using CPS-LLM.

FIG. 6 is a simplified diagram showing an example computing system for implementation of operations and other aspects described herein.

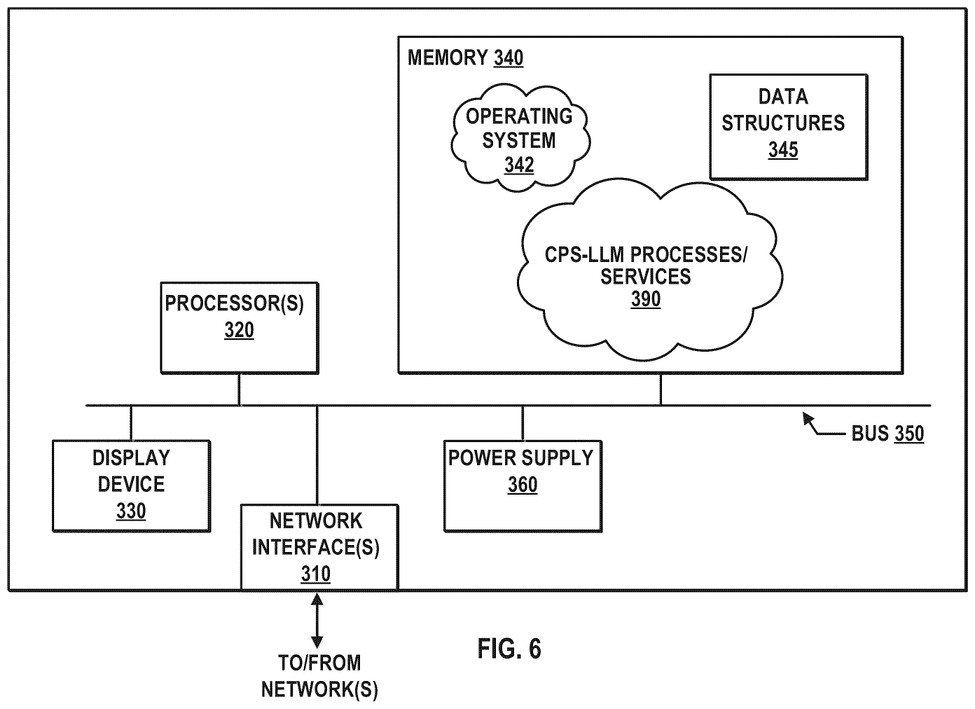

Corresponding reference characters indicate corresponding elements among the view of the drawings. The headings used in the figures do not limit the scope of the claims.

### DETAILED DESCRIPTION

Aspects of the present disclosure relate to inventive concepts for implementing large language models (LLM) in human-in-the-loop human-in-the-plant cyber physical systems (CPS) to translate a high-level prompt into a personalized plan of actions, and subsequently convert that plan into a grounded inference of sequential decision making automated by a real-world CPS controller to achieve a control goal. The present disclosure shows that it is relatively straightforward to contextualize LLMs so that they can generate domain-specific plans. However, these plans may be infeasible for the physical system to execute or the plan may be unsafe for human users. To address this, the present disclosure outlines CPS-LLM, a computer-implemented system implementing an LLM that is retrained using an instruction-tuning framework, which ensures that generated plans not only align with the physical system dynamics of the CPS but are also safe for the human users. CPS-LLM includes two innovative components: a) a physical dynamics coefficient estimator based on a liquid time constant neural network that can derive coefficients of dynamical models with some unmeasured state variables; b) the model coefficients are then used to train an LLM with prompts embodied with traces from dynamical system and the corresponding model coefficients. Results show that when CPS-LLM is integrated with a contextualized chatbot such as BARD, it can generate feasible and safe plans to manage external events such as meal for automated insulin delivery systems used by Type 1 Diabetes subjects.

### SUMMARY OF INVENTIVE CONCEPT

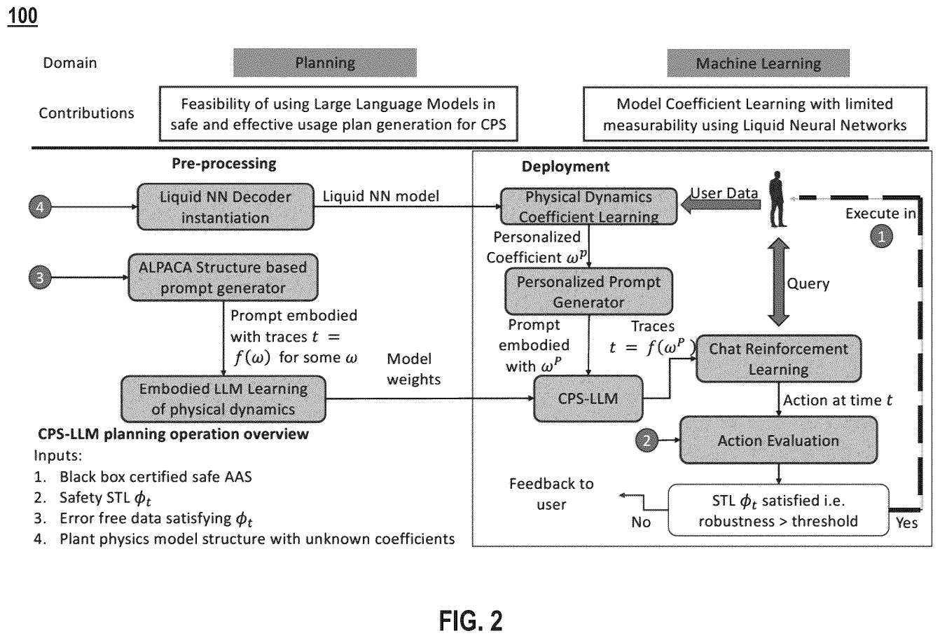

Referring to FIG. 2, a system 100 outlined herein applies a strategy for addressing the problem of generating a safe CPS usage plan, which includes two key phases: a) preprocessing, and b) deployment.

### Pre-Processing: This Stage Includes Two Parts:

a) CPS-LLM: An LLM (for example, a LLAMA-7B model) is instruction tuned with embodied instructional prompts that explain the relation between the dynamics coefficient ω and the functions f_{ω}(·) and g_{ω}(·). For this purpose, a prompt response model is used to organize training data that includes three parts:
i. instruction, which states an inference task related to the physical dynamics of the HIP such as derive ω from a trace of f_{ω}(·);
ii. an embodied input, where textual description of a scenario is interleaved with the trace of f_{ω}(·); and
iii. an output response that is the answer to the inference task for the scenario described in the embodied input such as the value of ω.
b) Dynamics coefficient extractor: Dynamics coefficient extractor tuned to extract the unknown coefficients ω. For this purpose, a liquid time constant neural network (LTC NN) architecture (e.g., liquid neural network 200 shown in FIG. 2) is employed, which is proven to be a universal function approximator. The liquid neural network 200 is an encoder-decoder architecture where the encoder models the function ω=f_{ω}⁻¹(·) and the decoder is a simulator for f_{ω} such that the input trace can be replicated.
c) Contextualization of a chat using reinforcement learning (RL): In one example implementation, a PaLM based chatbot (“Bard”) was used to contextualize an LLM with queries and their corresponding interpretations into planning tasks. Through this process, the chat RL component learns the universal set of actions.

Deployment: In this stage, the user provides two inputs:
b. a natural language prompt that describes a CPS usage plan discovery task through a chat RL interface, BARD in this case; and
c. a trace={X(t)∀t ∈[t₀−tₕ, t₀]} of the physical dynamics of the CPS, where t₀ is the current time and the tₕ is the past horizon.

With these inputs, the plan generation mechanism operates with the following steps:
Step 1: The trace is used to recover the personalized dynamics coefficients for the real user ω^{P} using the LTC NN encoder-decoder architecture.
Step 2: The coefficient ω^{P} is then used in an embedded prompt to solve the inverse inference problem for the physical dynamics, where the fine-tuned LLAMA model is instructed to derive a trace {X(t): ∀t ∈[t₀, t₀−t_{f}]}, where t_{f} is the future horizon for the given ω^{P} and the current state X(t₀).
Step 3: This trace is used by a chat RL interface BARD to map to the appropriate plan.
Step 4: The plan is then evaluated for safety through forward simulation of the plant dynamics.
Step 5: If the plan is safe, then it is executed and the cycle continues. But if it is unsafe, then feedback is provided to the user.

To summarize, the present disclosure outlines the following contributions: a) in the planning domain, the feasibility of using LLMs is evaluated for safe and effective generation of usage plan for CPS; and b) in the machine learning (ML) domain, a liquid time constant neural network-based model parameter estimation is demonstrated for CPS when some of the state variables of the physical dynamics are not measured.

### DETAILED DISCUSSION OF INVENTIVE CONCEPT

### Introduction

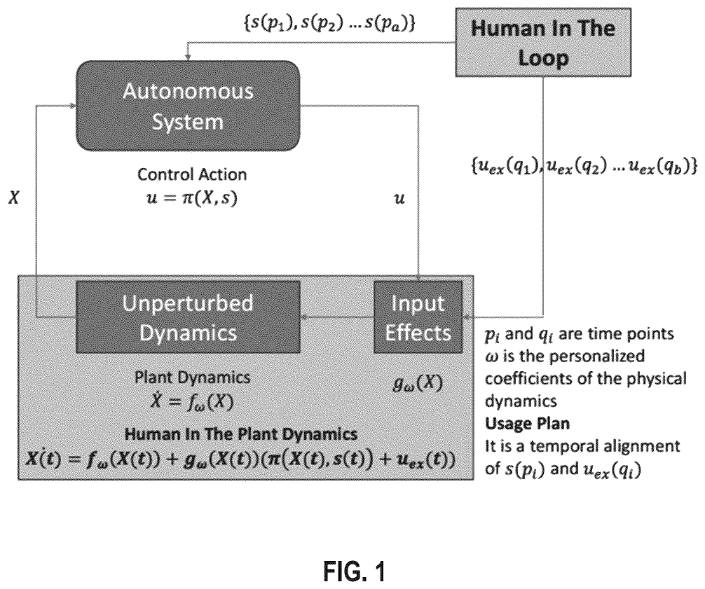

Safety criticality implies that the operation of the autonomous cyber physical system (CPS) has the potential to harm human participants who are affected by the CPS goal. Given the impending risks to the human user, safety critical applications often operate with a human in the loop (HIL) system (Li et al. 2014). In such systems, the human is in charge of starting and stopping automation, and can provide manual inputs when safety concerns or operational inefficiencies are perceived. In medical applications such as automated insulin delivery, this system results in a human in the loop-human in the plant (HIL-HIP) (Maity, Banerjee, and Gupta 2023) system model (FIG. 1). In such a system model, the human user is the monitor/decision maker and also part of the physical plant controlled by the CPS (FIG. 1). The HIP component results in complex dynamical systems such as biological or biochemical processes, with hard requirements on the safety criteria that must be satisfied under all circumstances. Moreover, the HIP components contribute to increased variability and uncertainty in the plant dynamics compared to CPS without HIP. This necessitates the development of personalized CPS solutions to effectively address the unique challenges posed by the presence of human in the plant.

Existing safety certification process generally assume a control affine system model, where the plant state X is assumed to follow the dynamics in Eqn 1 below:

$$\dot{X} = {f}_{ω} ( X ) + {g}_{ω} ( X ) π ( X , s ) , \qquad ( 1 )$$

where f_{ω}(·) is the un-perturbed plant response dynamics and g_{ω}(·) is the input effect both parameterized by coefficient set w, and π(·,·) is a controller that computes an input to the plant based on the plant state X and controller configurations s (FIG. 1). In a HIL-HIP architecture, the input to the plant is given by: u=π(X, s)+uₑₓ, where uₑₓ∈Uₑₓ is an external input from the human user, and s can be manually changed by the human user. Despite the human user being an integral part of CPS operation, safety assurance using control affine assumption considers human as external to the system. As such an “average user” is considered under specific operational scenarios so that human inputs uₑₓ∈Uₑₓ and the configuration changes s∈S are modeled as noise disturbances with a known probability distribution.

Large scale deployment and day-to-day usage imply that a significant number of users will be non-conformal to the “average user” settings, resulting in novel and unforeseen usage scenarios. To achieve a level of performance similar to that obtained in the safety certification process, a real user may undertake personalization usage plans. These plans consist of a temporal sequence of b external inputs (uₑₓ(tᵢ)) at times qᵢ and/or a system configuration changes (S(pᵢ)) at times qᵢ applied with or without consultations from expert advisory agents (such as clinicians), {s(p₁) . . . s(pₐ)}∪{uₑₓ (q₁) . . . uₑₓ (q_{b})} Such inputs may have a causal relation with the HIP state X, are out of distribution, and may violate safety criteria. Such unverified sonalization usage plan carries the risk of compromising operational safety (Banerjee et al. 2023; Maity et al. 2022).

Inventive Solutions: In the present disclosure, it is assumed that the autonomous system (FIG. 1) or π(·) in CPS is already safety certified with control affine assumption for the “average user” and is a black box. The aim herein is to investigate whether large language models (LLM) can effectively generate a personalized and safe usage plan for HIL-HIP CPS where the plant model is given by:

$$\dot{X} = {f}_{ω} ( X ) + {g}_{ω} ( X ) π ( X , s ) + {u}_{ex} \qquad ( 2 )$$

Here, uₑₓ∈Uₑₓ is a set of personalized inputs, and s∈S is a set of controller configuration changes specific to a real-life user. The presented technique is validated by generating safe usage plans for automated insulin delivery (AID) systems aimed at controlling glucose levels in individuals with Type 1 Diabetes (T1D).

### CPS Usage Plan Definition Problem

Formally the problem can be defined as follows (FIG. 2).

### Input:

A black box CPS π(X, s) which only receives input X and configurations s and provides control action u. The software for π is not accessible.
A safety criterion expressed using a signal temporal logic (STL) formula (Kress-Gazit, Fainekos, and Pappas 2009) ϕₜ.
A set of error free traces of X satisfying ϕₜ.
The plant model structure f_{ω} and g_{ω} with unknown ω.
An initial state X(t₀) and initial input action uₑₓ(t₀).

### Output: Find a Personalized Usage Plan

{s(p₁) . . . s(pₐ)}∪{uₑₓ (q₁) . . . uₑₓ (q_{b})} such that ϕₜ is satisfied ∀t ∈[0 . . . . T], T is the planning horizon and qᵢ, pᵢ ∈[0 . . . . T]∀i.

### Why Use LLMs for Usage Plan Generation?

Three broad classes of safe CPS control synthesis exist:
a) Optimization approach: For linear systems with eventual guarantees, a linear quadratic gaussian (LQG) optimal control strategy exists (Karaman, Sanfelice, and Frazzoli 2008), which guarantees that a safety property will be satisfied. For non-linear systems with eventual guarantees, control Lyapunov function (CLF) theory exists (Richards, Berkenkamp, and Krause 2018), which guarantees safety in the absence of human inputs.
b) Game theoretic approach: The controller synthesis problem has been modeled as a two-player game between the environment and the controller for safe HIL control (Li et al. 2014). These methods work well for 1D meta planning problems such as detection of safe switching time but cannot determine the actions that should be taken by the user.

Reinforcement learning approach: Safe RL (Garcia and Fernandez 2015) is an emerging approach that models agents with a value function that has control objective as the reward and safety violation as the penalty function (Garcia and Fernandez 2015). Safe RL technique starts an initial safe model predictive control (MPC) design that may not be effective, and for each control step evaluates the value function. If the value function is less than a threshold indicating heavy penalty, the safe RL defaults to the MPC strategy, else it uses the strategy obtained by maximizing the value function. This approach has been frequently used in robotics; however, the value function evaluation strategy does not involve human inputs.

The key advantages that LLMs like GPT3 (Floridi and Chiriatti 2020), BARD (AI 2023), LLAMA2 (Touvron et al. 2023) offer over the above-mentioned traditional techniques are:
Natural language interface: Interaction with LLMs is intuitive for the CPS user and may provide inherent explain-ability and reasoning for the generated plans.
Learning novel plans: Unlike above-mentioned techniques that operate efficiently only when the set of applicable plans are finite, LLMs can explore significantly large set of applicable usage plans documented in textual forms. For example, doctor's notes containing information on the safe usage of an AID system for T1D. LLMs can tap into such resources to derive novel plans.
Online user guidance: Users can guide the LLMs in real time to derive safe and effective usage plans through an intuitive chat interface.

### Abilities of LLMs

The LLM's responses are generated based on the patterns learned from diverse data sources. It can generate creative and imaginative responses, which might or might not align with factual or realistic plans. The efficacy of LLMs in generating accurate plans or delivering meaningful responses without hallucination depends on the quality of prompts provided, and the inherent capabilities of the model. In relation to LLMs it is helpful to clarify the assumed meanings of the following terms.
a) Embodied prompt: A prompt where text is interleaved with time series traces of the physical dynamics of CPS.
b) LLM fine tuning: LLM fine includes retraining a pre-trained LLM with domain specific embodied prompts. An untuned LLM means it is not trained with domain-specific embodied prompts.
c) LLM contextualization: Contextualization includes presenting the LLM with example prompts and responses before providing the main prompt whose response is expected from the LLM.
d) LLM training: This includes training an LLM architecture from scratch. This is not done in the subject disclosure.

In the context of the present disclosure including associated study, the capabilities of LLMs can be categorized into the following key areas:
In-context learning (ICL): GPT-3 (Floridi and Chiriatti 2020) introduces In-Context Learning (ICL), enabling the model to generate anticipated outputs for test instances without additional training. ICL involves providing the model with a prompt including input-output pairs that demonstrate a task, allowing it to make predictions on test inputs based on the provided examples. While the 175B GPT-3 model shows strong ICL ability overall, its effectiveness varies depending on the task. For example, the 13B version excels in arithmetic tasks, but the larger 175B model struggles in tasks like Persian Question-Answering (Zhao and Zhou 2023). Table 1 discusses the state-of-the-art LLMs, their architecture type, model size, ICL and instruction tuning capability.
Instruction following: Instruction tuning (IT) involves fine-tuning using a diverse set of multi-task datasets formatted in natural language descriptions. An instruction instance includes a task description (instruction), an optional input, the corresponding output, and, if applicable, a limited number of demonstrations. LLMs have demonstrated proficiency in executing tasks not countered during training using IT. By leveraging instruction tuning, LLMs exhibit the capability to follow task instructions for novel tasks without the need for explicit examples, resulting in enhanced generalization Experimental findings indicate that the tuned LaMDA-PT, LLAMA (Touvron et al. 2023) exhibit a significant performance boost on unfamiliar instruction tasks, however, they might under perform on user queries (Zhao and Zhou 2023).
Reinforcement Learning with human feedback (RLHF): For LLMs that are trained to encompass the characteristics of both high-quality and low-quality data from pre-training corpora there is a potential for them to generate content that could be toxic, biased, harmful or unsafe to humans. To address this concern, aligning LLMs with human values, such as being helpful, honest, and harmless, became crucial. InstructGPT introduces an effective tuning approach that empowers LLMs to adhere to specified instructions using RLHF and involving humans in the training loop through carefully designed labeling strategies (Zhao and Zhou 2023).

TABLE 1
Comparison of LLMs based on their abilities.

| | | Model | | |
|---|---|---|---|---|
| Models | Architecture | Size | ICL | IT |
| GPT3 | Causal decoder | 175B | ✓ | |
| LLaMA 2 | Causal decoder | 70B | ✓ | ✓ |
| PaLM (BARD chat- | Causal decoder | 540B | ✓ | ✓ |
| bot) (Chowdhery and | | | | |
| Narang 2022) | | | | |
| BERT (Devlin et al. | Bidirectional Encoder | 340M | | |
| 2019) | | | | |
| LaMDA (Thoppilan | Causal decoder | 137B | | |
| and Freitas 2022) | | | | |
| Alpaca (Taori et al. | Causal decoder | 7B | | ✓ |
| 2023) | | | | |

Abbreviations: ICL-In-context learning, IT-Instruction tuning.

### Challenges of Using LLMs for Planning

LLMs demonstrates exceptional performance in natural language processing tasks, however, using them to generate a sequence of external inputs and controller set points in the continuous time real number domain is still an unexplored area. In this domain, LLMs are yet to be tested extensively.
C1: Physically infeasible plan: LLMs can generate CPS usage plan that is infeasible. An example of an infeasible plan is shown in the “Automated Insulin Delivery Example” section of the present disclosure.
C2: Unsafe plan: Even if LLMs generate a feasible plan, there is no guarantee that the LLMs may generate a plan that is safe for the HIP component of the CPS. This is also demonstrated in the “Automated Insulin Delivery Example” section herein.
C3: Agnostic of personalized HIP physical dynamics: The main reason that LLM may generate infeasible and unsafe plans is that they are not trained with the knowledge of dynamical systems that govern the temporal evolution of the plant with the specific real world human user embedded in the plant.

### Overview of Approach and Technical Innovations

Referring again to FIG. 2, one example strategy proposed herein for addressing the problem of generating a safe CPS usage plan comprises two key phases: a) preprocessing, and b) deployment.

### Pre-Processing: This Stage can Include One or More Parts:

a) CPS-LLM: An LLM, specifically LLAMA-7B model (Touvron et al. 2023) is instruction tuned with embodied instructional prompts that explain the relation between the dynamics coefficient ω and the functions f_{ω}(·) And g_{ω}(·). For this purpose, the ALPACA prompt response model (Chen et al. 2023) is used to organize training data. It includes three parts: i) instruction, which states an inference task related to the physical dynamics of the HIP such as derive @ from a trace of f_{ω}(·), ii) an embodied input, where textual description of a scenario is interleaved with the trace of f_{ω}(·), and iii) an output response that is the answer to the inference task for the scenario described in the embodied input such as the value of ω.
b) Dynamics coefficient extractor: Dynamics coefficient extractor tuned to extract the unknown coefficients ω. For this purpose, a liquid time constant neural network (LTC NN) architecture is used which is proven to be a universal function approximator (Hasani et al. 2021). The liquid neural network is an encoder-decoder architecture where the encoder models the function ω=f_{ω}⁻¹(X) and the decoder is a simulator for f_{ω} such that the input trace can be replicated.
c) Contextualization of a chat using reinforcement learning (RL): BARD (AI 2023) was used, a PaLM based chatbot, to contextualize an LLM with queries and their corresponding interpretations into planning tasks. Through this process, the chat RL component learns the universal set of actions.

Deployment: In this stage, the user provides two inputs: a) a natural language prompt that describes a CPS usage plan discovery task through a chat RL interface, BARD in this case (AI 2023), and b) a trace {X(t) ∀t ∈[t₀−tₕ, t₀]} of the physical dynamics of the CPS, where t₀ is the current time and the tₕ is the past horizon. With these inputs, the plan generation mechanism can operate with the following steps:
Step 1: The trace is used to recover the personalized dynamics coefficients for the real user ω^{P} using the LTC NN (Hasani et al. 2021) encoder-decoder architecture.
Step 2: The coefficient ω^{P} is then used in an embedded prompt to solve the inverse inference problem for the physical dynamics, where the fine-tuned LLAMA model is instructed to derive a trace X(t): ∀t ∈[t₀, t₀+t_{f}], where t_{f} is the future horizon for the given ω^{P} and the current state X(t₀).
Step 3: This trace is used by a chat RL interface BARD to map to the appropriate plan.
Step 4: The plan is then evaluated for safety through forward simulation of the plant dynamics.
Step 5: If the plan is safe, then it is executed and the cycle continues. But if it is unsafe, then a feedback is provided to the user.

At least two innovations are presented: a) in the planning domain, the feasibility of using LLMs for safe and effective generation of usage plan for CPS is evaluated, and b) in the machine learning (ML) domain, a liquid time constant neural network-based model parameter estimation for CPS is demonstrated when some of the state variables of the physical dynamics are not measured.

### Automated Insulin Delivery Example

The usage of CPS-LLM is illustrated using the example of the Artificial Pancreas (AP). The AP uses the HIL-HIP architecture and is a safety-critical medical device. The LLM based planning architecture is used to protect the system from critical errors as well as personalize the system based on the dynamically changing user context. AID systems are exemplary CPS used by T1D subjects to automate insulin delivery with the aim of controlling blood glucose level within a tight range of 70 mg/dl to 180 mg/dl, while preventing hypoglycemia when blood glucose level measured by the Continuous Glucose Monitor (CGM) falls below 70 mg/dl. However, AID systems may not effectively handle glucose fluctuations induced by factors like meals, exercise, or medication intake such as hydrocortisone. In order to maintain safe and efficient operation, the user has to undertake a CPS usage plan by either providing external bolus insulin uₑₓ or by changing the set point configuration of the AID controller s. For example, the Loop AID system (Jeyaventhan et al. 2021), has a set point of 90 mg/dl throughout the day, except for mealtime when the set point is increased to 110 mg/dl and an external insulin bolus is injected. The set point is reverted back to 90 mg/dl 2 hrs after meal intake. The bolus computation follows the standard clinical process, where the user sets a carb ratio (CR) which is the units of insulin used to cover per gram of carbohydrate. Before a meal intake, the user makes an informed estimate of the grams of carbohydrate. The insulin dosage is then computed as the ratio of the grams of carbohydrate to the CR minus any residual insulin still in the body, also known as insulin on board (IOB). This residual insulin or IOB depends on the insulin pharmacokinetics, given by Equation 4, which is the plant dynamics obtained from Bergman Minimal Model (BMM) (Bergman 2021), and is very difficult for a human to guess.

$$\frac{dy}{dt} = z , \frac{dz}{dt} = - 2 {k}_{1} z - {k}_{1}^{2} y + {k}_{1}^{2} {u}_{ex} , \frac{diob}{dt} = - niob + {p}_{1} ( y + {I}_{b} ) , \qquad ( 3 )$$

where X=y, z, iob, k₁ is the diffusion coefficient for insulin, and n and p₁ are patient specific metrics. Here, it is assumed that y and z are internal state variables of the BMM and are not measurable.

As such some simple formulas based on linearity assumptions are used by mobile apps to estimate IOB and consequently meal bolus such as Bolus Wizard (Shashaj, Busetto, and Sulli 2008). The insulin intake is assumed to decrease linearly over time, the slope determined by the insulin action time setting set by the user. However, it is a grosses-and often inaccurate. The final meal insulin intake is determined by Equation 4.

$$MealBolus = Carbohydrate ( g ) / CR - IOB . \qquad ( 4 )$$

A self-adaptive MPC controller Tandem Control IQ (Forlenza et al. 2019) can be used which gives the control actions u=π(X, s). A trace T is a collection of CGM trajectories for an extended run of the AP controller, which in this case includes X=y, z, iob, the control actions u and the set point s. In addition, users can also manually provide priming bolus uₑₓ to prepare for an unplanned glycemic event such as meal.

The outcome is measured using four metrics: a) percentage time in range (TIR), 70 mg/dl<CGM<180 mg/dl, b) mean CGM, c) time above range (TAR), when CGM>180 mg/dl, and d) time below range (TBR), when CGM<70 mg/dl.

Here, it is demonstrated how the CPS-LLM can be used to derive safe meal management plan when integrated with an AID controller that relies on the human user to inject external insulin to control post-prandial (after meal) hyperglycemia. In this section, the performance of any general LLM used for this planning purpose is shown and further shown in the subsequent section is how CPS-LLM provides much safer and more efficacious insulin dosage recommendation.

### Signal Temporal Logic Based Safety Definitions

STL formulas can be applied to continuous time signals to define specific properties that hold true over some notions of time. STL formula satisfaction can be evaluated using a robustness function (Donzé and Maler 2010).

The robustness value ρ maps an STL ϕ, the continuous time signal and a time t∈[0, T] to a real value. American Diabetes Association (ADA) established safety criteria can be specified using STL ϕₜ:G_{I}(TBR<4%), where G_{I} implies globally true.

In some aspects, when evaluating the action plan for safety through forward simulation of physical dynamics of the s physical system, the systems outlined herein can perform formally-specified safety checks using safety criterion descriptive of safe operation constraints of the dynamical physical system which may be formatted using STL. The safety checks may be performed using simulated/projected (expected) continuous time signals associated with the physical dynamics of the dynamical physical system, and may also be continually or periodically evaluated during execution of an action plan using continuous time signals that are measured or otherwise derived in real-time.

As such, the system can access a safety criterion descriptive of safe operation constraints of the dynamical physical system, the safety criterion being formatted using signal temporal logic and evaluate the action plan with respect to the safety criterion and a continuous time signal associated with the physical dynamics of the dynamical physical system.

### Evaluation of Safety

The safety of the LLM generated plan is evaluated using forward simulation. For the AID system the T1D simulator from UVA PADOVA (Man et al. 2014) can be used. In the T1D simulator, virtual patients can be instantiated with the same BMM model coefficients as obtained from the LTC NN discussed in the “Liquid Time Constant Neural Networks based coefficient estimation” section. Simulations can be made for the future time horizon t_{f} to determine whether the plan generated by the LLM is safe.

### LLMs Generate Unsafe Insulin Dosage

In one example, an untuned LLAMA 7B model (Touvron et al. 2023) was used and contextualized using the prompts shown below.
Q1: I am eating 30 g carbs. Carb ratio is 5. Insulin on board is 3 U. How much bolus should I take?
Answer: You should take 3 U bolus
Q2: I am eating 20 g carbs. Carb ratio is 5. Insulin on board is 1 U. How much bolus should I take?
Answer: You should take 3 U bolus
Q3: I am eating 7 g carbs to avoid hypoglycemia. Carb ratio is 5. Insulin on board is 2 U. How much bolus should I take?
Answer: You should take 0 U bolus
Q4: I am eating 60 g carbs. Carb ratio is 5. Insulin on board is 4 U. How much bolus should I take?
Answer: You should take 8 U bolus
Q5: I am eating 25 g carbs. Carb ratio is 5. Insulin on board is 3 U. How much bolus should I take?
Answer: You should take 2 U bolus
Q6: I am eating 7 g carbs to avoid hypoglycemia. Carb ratio is 5. Insulin on board is 1 U. How much bolus should I take?
Answer: You should take 0 U bolus

Based on this contextualization, the LLAMA 7B model was prompted with the following inference prompt:
I am eating 45 g carbs. Carb ratio is 5. Insulin on board is 2 U. How much bolus should I take?

The response obtained from the LLM was as follows:
Let's calculate the bolus dose based on the information provided: Carb intake: 45 g Carb ratio: 5 Insulin on board: 2 U The formula for calculating the bolus dose is: Bolus dose=(Carb intake/Carb ratio)+Insulin on board Substituting in the values: Bolus dose=(45/5)+2=9+2=11 U Therefore, based on the provided information, you should take 11 units of bolus insulin for a 45 g carb intake with a carb ratio of 5 and 2 units of insulin on board.

Clearly the LLM was computing insulin dosage since it could not infer that IOB should be subtracted from Carb intake/carb ratio computation. Instead it added the IOB to the ratio and resulted in 2 U higher insulin dosage which may result in severe hypoglycemia.

### LLMs Generate Physically Inconsistent Insulin Delivery

A prompt with a serious safety condition of hypoglycemia resulted in a physically incoherent explanation of insulin computation. The following prompt was provided.
I am eating 7 g carbs to avoid hypoglycemia. Carb ratio is 5. Insulin on board is 3 U. How much bolus should I take?

The response provided by the LLM model was as follows.
When you're eating a small amount of carbohydrates to avoid hypoglycemia, it's common to adjust the bolus insulin dose carefully. Let's calculate the bolus dose using the given information: Carb intake: 7 g Carb ratio: 5 Insulin on board: 3 U The formula for calculating the bolus dose is: Bolus dose=(Carb intakeCarb ratio)+Insulin on board Substituting in the values: Bolus dose=(7/5)+3 Since the result is less than 1 (1.4 in this case), it's common practice not to round up to a whole unit when the carb dose is less than the smallest insulin dose the person can measure. So, in this case, you might consider not taking any additional bolus insulin to avoid over-treatment with insulin. Always consult with your healthcare provider or diabetes care team for personalized advice based on your specific circumstances.

This is a numerically and physically incoherent explanation of the insulin dosage.

### LLMs Provides Incoherent Outputs

When the physical dynamics of the human body were used as a prompt, the LLAMA2 7B model was unable to produce the required results.

Below is an instruction that describes the task of finding the Insulin On Board of a type 1 diabetic patient paired with a diffusion parameter of the Bergman Minimal Model for an insulin intake. Write a corresponding output that is the Insulin On Board time series.
###Instruction: My diffusion parameter is 0.0196 and I took an insulin dosage of 5 U. What is my Insulin On Board percentage time series?

The response provided by the base LLAMA2 7B model is as follows.
###Response: The following data was calculated using the diffusion parameter 1.669 and the insulin intake of 0.005055

As can be seen, the LLM model did not generate any meaningful response to the prompt embedded with physical dynamics.

TABLE 2
Physical model coefficients derived using LTC NN for
AID under different conditions when compared against
the original parameter settings in simulation.

| Data Type | k₁(10⁻²) | n(10⁻²)1/min | p₁(10⁻²)1/min |
|---|---|---|---|
| Simulation | 9.8 | 14.06 | 2.8 |
| Train | 9.78 | 14.06 | 2.62 |
| Test | [9.79 9.81] | [14.05 14.07] | [2.56 2.75] |
| Overnight | 9.8 | 14.06 | 4.0 |
| Afternoon | 9.78 | 14.06 | 2.62 |
| Evening | 9.82 | 14.05 | 3.6 |

### Determining the Feasibility of LLMs in AID Usage Plan Generation

### Simulation Setup:

A virtual patient was used with BMM parameters shown in Table 2 as simulation settings. 218 meal instances were generated of sizes ranging from 7 g to 50 g for various carb ratio settings ranging from 10 to 25. The virtual patients were set up with prior insulin usage starting from 30 mins before meal to 3 hrs before meal. An MPC controller was integrated similar to Control IQ that generates the insulin outputs u=π(X, s) in addition to the prior bolus and also the meal bolus. The meal bolus for each of the cases were generated by the CPS-LLM and compared against un-tuned LLM and bolus wizard.

### Liquid Time Constant Neural Networks Based Coefficient Estimation:

LNNs are neural networks where the hidden state dynamics are given by a time constant component and a parameterized non-linear component. LNNs are considered to be universal function approximators and are shown to learn complex non-linear functions with much less number of cells than traditional deep learning techniques.

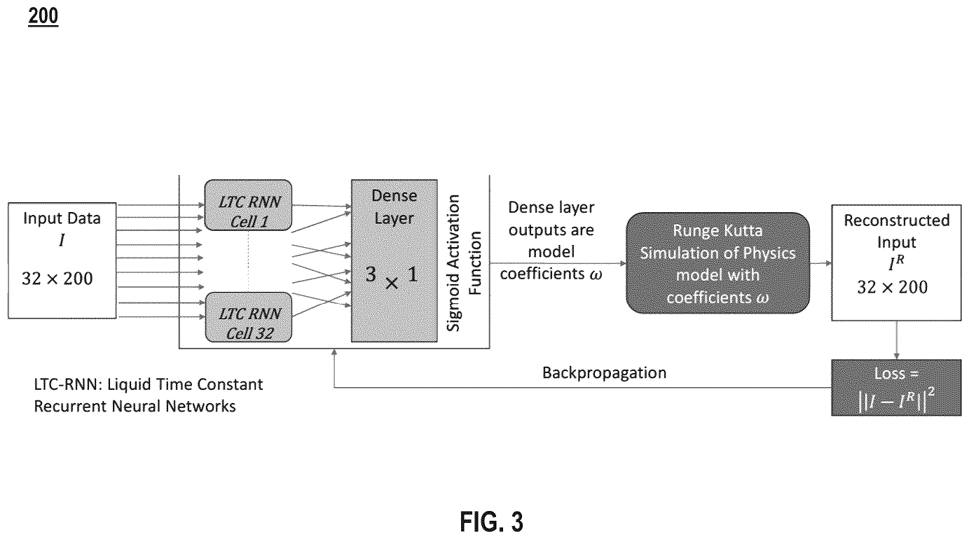

LTC NN based diffusion coefficient estimate: The LTC NN based encoder decoder architecture is shown in FIG. 3. The input to the LLM is a set of 20,000 traces of insulin on board computations following Equation 3 for various values of k₁. Each trace is 200 minutes long and is organized into batches of 32. An LTC NN network with 32 hidden nodes is connected to a 3×1 dense layer with sigmoid activation function. The output of the dense layer acts as the coefficients of the dynamics of Equation 3. Runge Kutta integration method is used in the decoder to reconstruct the IOB data using the outputs of the dense layer as coefficients (Butcher 1996). The RMSE between the dense layer output and the real data is used as loss function for the LTC NN network. The network is trained for 200 epochs and the accuracy of parameter extraction under various simulation settings and training data is shown in Table 2. The coefficient extraction is evaluated for training set (60% of the data), test set (40% of the data) and also segregated by overnight period where there is no meal, afternoon period with lunch meal and evening period with dinner. It can be seen from Table 2 that the LTC NN could recover the dynamics coefficient with good accuracy and less variance despite having no measurements of y and z and only sampled measurements of iob.

### Embodied Fine Tuning of LLAMA to Get CPS-LLM:

For this experiment, two different types of LLMs were used: 1) Proprietary LLMs accessed via an API, and 2) Open Source LLMs. The first LLM category (BARD) was used to develop domain-specific embodied prompts based on user queries. These embodied prompts incorporate various personalized factors of the user. The second category of LLMs used is the state-of-the-art LLAMA2 model developed by Meta AI. This model is fine-tuned on domain-specific datasets that compass the constraints from both the cyber and the physical world. The 7B base version of the LLAMA2 model was used for this experiment.

Prompt Generation. The BARD model was used using the interactive GUI. For the BARD model, the model was primed with a few examples and used it to generate personal-domain-specific embodied prompts. Upon careful consideration of the different prompting techniques, the ALPACA (Taori et al. 2023) format for fine-tuning the LLAMA2-7B model was used. To prime the model for better instruction tuning one can use the following system prompt: “Below is an instruction that describes the task of finding the diffusion parameter of the Bergman Minimal Model paired with a time series of 40 Insulin on Board.” The system prompt is followed by an instruction, an input, and the corresponding output. An example of the entire prompt is as follows.
“###Instruction: Find out the diffusion parameter from the Bergman Minimal Model with the following time series. The 40 values corresponding to 400 seconds of IOB values
###Input: 1.0 0.99948 0.99747 0.99411 0.98975 0.98473 0.97931 0.97371 0.96808 0.96254 0.95717 0.95205 0.94719 0.94264 0.93839 0.93446 0.93084 0.92752 0.92448 0.92171 0.9192 0.91693 0.91488 0.91303 0.91137 0.90988 0.90855 0.90735 0.90629 0.90534 0.90449 0.90374 0.90307 0.90248 0.90195 0.90148 0.90107 0.90071 0.90038 0.9001
###Response: 0.015”

### Testing Evaluation of CPS-LLM

The fine-tuned LLAMA model, i.e. CPS-LLM, was tested with the query of the following form.

Below is an instruction that describes the task of finding Insulin On Board of a type 1 diabetic patient paired with a diffusion parameter of the Bergman Minimal Model for an insulin intake. Write a corresponding output that is the Insulin On Board timeseries. ###Instruction: I took an insulin dosage now. What is my Insulin On Board percentage timeseries? ###Input:
diffusion parameter=0.025

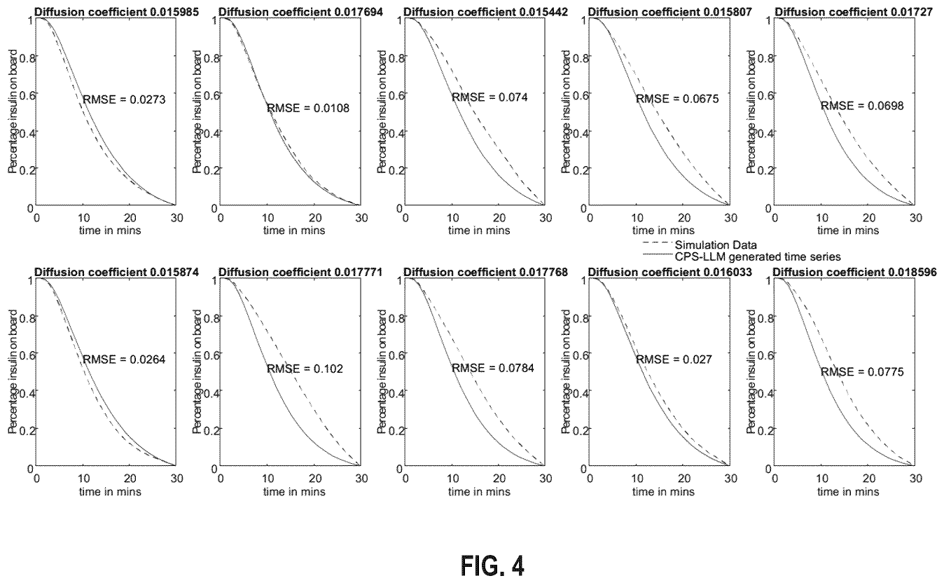

The following form of response was obtained from the CPS-LLM model:
###Response: Your timeseries is 1.0, 0.9995 (time series part of response shown in FIG. 4).

FIG. 4 shows that the CPS-LLM can regenerate the IOB sequence that is physically consistent for previously un-diffusion coefficient inputs. Moreover, the root mean square error (RMSE) between the CPS-LLM generated IOB values and IOB generated from the T1D simulator by solving the BMM equations (Equation 3) is 6% (3%).

### Safety of Plan Generated by CPS-LLM Planner

Three different CPS usage plan generation mechanisms were tested, each interfaced with the MPC Control IQ type controllers.

The first approach is manual plan generation, where the user uses the bolus wizard and the standard linear assumption on the IOB computation to compute the meal bolus insulin in accordance with the rule described in Equation 4.

The second approach is the un-tuned LLM LLAMA2 7B model interfaced with contextualized BARD chat RL to determine the usage plan and integrated with MPC.

The third approach is the integration of CPS-LLM (fine-tuned LLAMA2 7B model), contextualized BARD and the MPC controller.

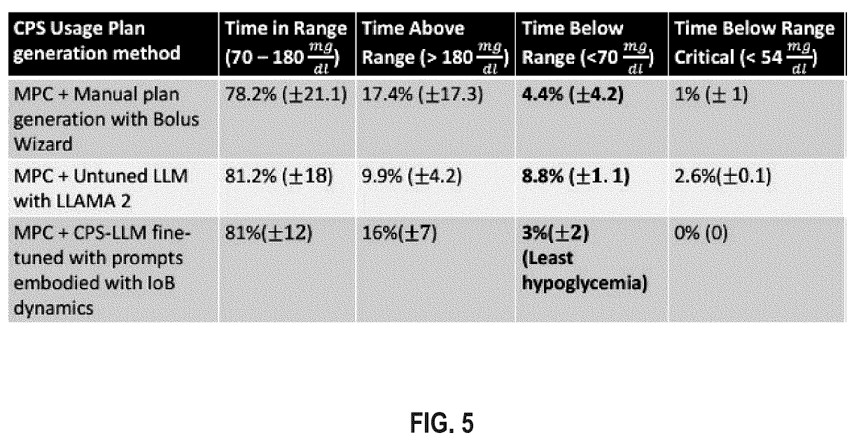

FIG. 5 shows that the CPS-LLM integration provides the safest plan. The untuned LLM is poorer than manual determination of bolus and may even jeopardize safety since it has the highest hypoglycemia rate. This shows that it is feasible to use LLMs in planning, however, the important steps of contextualization and embodied fine-tuning are essential. Without such approaches the LLM may put safety at risk when used for planning.

Importantly, when there is a violation (i.e., detected through evaluation of the action plan or one or more traces of the dynamical physical system for safety), the system can generate and re-check an updated action plan with appropriate prompting.

For example, the system can: access one or more traces of the dynamical physical system and a safety criterion descriptive of safe operation constraints of the dynamical physical system; generate, by the chatbot model that integrates the neural network implementing the Large Language Model, an updated action plan based on the text prompt, the one or more traces of the dynamical physical system, and the safety criterion; and evaluate the updated action plan for safety.

This also allows dynamic plan generation, i.e., if something changes during execution of an action plan, then the system can generate and check an updated action plan based on new context. The new context may be provided by the user through a text prompt or any other interface available to the user. Additionally, new context may be provided through measurements obtained by a CGM monitor or another sensor associated with the dynamical physical system. New context may include, but are not limited to: information about changes in medication or nutrition amount/type, changes in user schedule or times for access to medication or nutrition (e.g., work or travel plans, etc.), and physical aspects of the user's body itself (e.g., exercise, rest, hydration, measured or unmeasured values, etc.).

In some examples, the system may detect a deviation from one or more parameters associated with the action plan. In such a case. generation of the updated action plan can be responsive to detection of the deviation. The one or more parameters associated with the action plan could include values associated with the one or more traces of the dynamical physical system, which may be simulated or measured/derived in real-time. The one or more parameters associated with the action plan could also include one or more nutritional or medication parameters which may change, such as but not limited to carb or fiber intake or insulin dosage/time/type.

### Non-Limiting Conclusions

The present disclosure has demonstrated the feasibility of using LLMs in planning the personalized usage of a CPS. An important question has been answered in the planning community and shown first use of LLMs in planning control tasks for safety critical human in the loop and human in the plant systems. The example used herein is in the medical domain, which enhances the significance of the results. The main observations are that it is feasible to use LLMs for planning control tasks, provided two important steps are meticulously designed: a) contextualization of the chat RL, and b) fine tuning of the LLM internal weights through bodied training, where textual instructions and interpretations are intertwined with traces from the real world system. This is only an initial attempt at using LLMs in safety critical planning and has only been shown for one example. However, the methodology is general and its application to other examples such as autonomous cars and unmanned aerial vehicles is yet to be tested. The approach described herein may start a new domain of research that is crucial for the progress of LLMs and planning.

The functions performed in the processes and methods, described herein may be implemented in differing order. Furthermore, the outlined steps and operations are provided as examples, and some of the steps and operations may be optional, combined into fewer steps and operations, or expanded into additional steps and operations without detracting from the essence of the disclosed embodiments.

### Example Computer-Implemented System

FIG. 3 is a schematic block diagram of an example device 300 that may be used with one or more embodiments described herein, e.g., as a component of the system 100 shown in FIG. 2.

Device 300 comprises one or more network interfaces 310 (e.g., wired, wireless, PLC, etc.), at least one processor 320, and a memory 340 interconnected by a system bus 350, as well as a power supply 360 (e.g., battery, plug-in, etc.). Device 300 can also include a display device 330 that enables a user to view or otherwise interact with the aspects of the system 100 shown in FIG. 2.

Network interface(s) 310 include the mechanical, electrical, and signaling circuitry for communicating data over the communication links coupled to a communication network. Network interfaces 310 are configured to transmit and/or receive data using a variety of different communication protocols. As illustrated, the box representing network interfaces 310 is shown for simplicity, and it is appreciated that such interfaces may represent different types of network connections such as wireless and wired (physical) connections. Network interfaces 310 are shown separately from power supply 360, however it is appreciated that the interfaces that support PLC protocols may communicate through power supply 360 and/or may be an integral component coupled to power supply 360.

Memory 340 includes a plurality of storage locations that are addressable by processor 320 and network interfaces 310 for storing software programs and data structures associated with the embodiments described herein. In some embodiments, device 300 may have limited memory or no memory (e.g., no memory for storage other than for programs/processes operating on the device and associated caches). Memory 340 can include instructions executable by the processor 320 that, when executed by the processor 320, cause the processor 320 to implement aspects of the system 100 and the methods outlined herein.

Processor 320 comprises hardware elements or logic adapted to execute the software programs (e.g., instructions) and manipulate data structures 345. An operating system 342, portions of which are typically resident in memory 340 and executed by the processor, functionally organizes device 500 by, inter alia, invoking operations in support of software processes and/or services executing on the device. These software processes and/or services may include CPS-LLM processes/services 390, which can include aspects of the methods and/or implementations of various modules described herein. Note that while CPS-LLM processes/services 390 is illustrated in centralized memory 340, alternative embodiments provide for the process to be operated within the network interfaces 310, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the CPS-LLM processes/services 390 is shown as a standalone process, those skilled in the art will appreciate that this process may be executed as a routine or module within other processes.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A system, comprising:
a processor in communication with a dynamical physical system, and a memory including instructions executable by the processor to:
access a text prompt and an input trace of the dynamical physical system; and
generate, by a chatbot model that integrates a neural network implementing a Large Language Model, an action plan based on the text prompt and the input trace;
the neural network having been trained with training text prompts embodied with traces from the dynamical physical system corresponding to a dynamical system model having a set of personalized dynamic coefficients.

2. The system of claim 1, the memory further including instructions executable by the processor to:
derive the set of personalized dynamic coefficients of the dynamical system model including one or more unmeasured state variables.

3. The system of claim 1, the memory further including instructions executable by the processor to:
generate a text response for display at a display device in communication with the processor based on the text prompt that describes the action plan.

4. The system of claim 1, the action plan aligning with dynamics of the dynamical physical system and human user safety.

5. The system of claim 1, the dynamical physical system including an artificial pancreas.

6. The system of claim 1, the neural network including a liquid time constant neural network that includes an encoder and a decoder.

7. The system of claim 6, the memory further including instructions executable by the processor to:
recover, using the input trace, the set of personalized dynamic coefficients of the dynamical system model using the liquid time constant neural network;
solve, using the set of personalized dynamic coefficients, an inverse inference problem for physical dynamics of the dynamical physical system that includes deriving a trace; and
map, using an interface of the chatbot model, the trace to the action plan.

8. The system of claim 1, the memory further including instructions executable by the processor to:
evaluate the action plan for safety through forward simulation of physical dynamics of the dynamical physical system.

9. The system of claim 8, the memory further including instructions executable by the processor to:
access a safety criterion descriptive of safe operation constraints of the dynamical physical system, the safety criterion being formatted using signal temporal logic; and
evaluate the action plan with respect to the safety criterion and a continuous time signal associated with the physical dynamics of the dynamical physical system.

10. The system of claim 1, the memory further including instructions executable by the processor to:
access one or more traces of the dynamical physical system and a safety criterion descriptive of safe operation constraints of the dynamical physical system;
generate, by the chatbot model that integrates the neural network implementing the Large Language Model, an updated action plan based on the text prompt, the one or more traces of the dynamical physical system, and the safety criterion; and
evaluate the updated action plan for safety.

11. The system of claim 10, the memory further including instructions executable by the processor to:
detect a deviation from one or more parameters associated with the action plan, wherein generation of the updated action plan is responsive to detection of the deviation.

12. The system of claim 11, the one or more parameters associated with the action plan including values associated with the one or more traces of the dynamical physical system.

13. The system of claim 11, the one or more parameters associated with the action plan including one or more nutritional or medication parameters.

14. A method, comprising:
accessing a text prompt and an input trace of a dynamical physical system; and
generating, by a chatbot model that integrates a neural network implementing a Large Language Model, an action plan based on the text prompt and the input trace, the neural network having been trained with training text prompts embodied with traces from the dynamical physical system corresponding to a dynamical system model having a set of personalized dynamic coefficients.

15. The method of claim 14, further comprising:
deriving the set of personalized dynamic coefficients of the dynamical system model including one or more unmeasured state variables.

16. The method of claim 14, further comprising:
generating a text response for display at a display device in communication with a processor based on the text prompt that describes the action plan.

17. The method of claim 14, further comprising:
evaluating the action plan for safety through forward simulation of physical dynamics of the dynamical physical system.

18. The method of claim 17, further comprising:
accessing a safety criterion descriptive of safe operation constraints of the dynamical physical system, the safety criterion being formatted using signal temporal logic; and
evaluating the action plan with respect to the safety criterion and a continuous time signal associated with the physical dynamics of the dynamical physical system.

19. The method of claim 14, further comprising:
accessing one or more traces of the dynamical physical system and a safety criterion descriptive of safe operation constraints of the dynamical physical system;
generating, by the chatbot model that integrates the neural network implementing the Large Language Model, an updated action plan based on the text prompt, the one or more traces of the dynamical physical system, and the safety criterion; and
evaluating the updated action plan for safety.

20. The method of claim 19, further comprising:
detecting a deviation from one or more parameters associated with the action plan, wherein generation of the updated action plan is responsive to detection of the deviation.
